(19) United States
(12) Patent Application Publication
(10) Pub. No.: US 2004/0019340 A1
(43) Pub. Date: Jan. 29, 2004

(54) Absorbent article having a surface energy gradient between the topsheet and the acquisition distribution layer

(75) Inventor: Robert K. McBride, Jasonville, IN (US)

Correspondence Address: FISH & RICHARDSON P.C., 5000 BANK ONE CENTER, 1717 MAIN STREET, DALLAS, TX, 75201, US

(73) Assignee: TREDEGAR FILM PRODUCTS CORPORATION

(21) Appl. No.: 10/202,253

(22) Filed: Jul. 23, 2002

(51) Int. Cl. A61F 13/15; A61F 13/20

(52) U.S. Cl. 604/378

(57) ABSTRACT

An absorbent article has a topsheet, an absorbent core, and an acquisition distribution layer between the topsheet and the core. The topsheet has an affinity for water and is therefore philic, not phobic. The acquisition distribution layer is more philic than the topsheet, thereby creating a surface energy gradient between the topsheet and the acquisition distribution layer.

The topsheet is preferably a formed film but may be nonwoven. The acquisition distribution layer is preferably of a high void volume type formed film but may be a standard formed film. The affinity to water of either the topsheet or the acquisition distribution layer may be created using surfactants, corona treatment, material selection or a combination of the above.

### BACKGROUND OF THE INVENTION

1. Technical Field of the Invention

This invention relates to absorbent articles such as sanitary napkins, disposable diapers, adult incontinence briefs, and the like. More particularly, the present invention relates to absorbent articles which have a topsheet, an absorbent core, and an acquisition distribution layer between the topsheet and the absorbent core.

2. Description of Related Art

The most generic design of an absorbent article is an absorbent core adjacent a topsheet facing the user and adjacent a back sheet on the opposite side. The absorbent core absorbs the bodily fluids or discharge. The topsheet controls fluid flow into and out of the absorbent core and masks the core from the user. The backsheet prevents fluid flow from the absorbent core on to the user's garments or bed linens.

These three basic items may be adjusted to meet the particular needs of the user. For example, the thickness of the absorbent core may be reduced for low fluid volume applications. Super absorbent polymers may be added to the core to drastically improve its performance. The material and texture of the topsheet may be adjusted to produce a more cloth-like feel, a silky feel, or a cottony soft feel or to produce greater masking depending on the customers' preference. In this regard, a nonwoven material or a formed film may be chosen depending on the application or consumer preference. The backsheet may be made breathable or more cloth-like if that is desired.

Topsheets were originally nonwoven materials which were accepted for their soft feel. However, nonwoven topsheets did not prevent rewet. Rewet is felt when a core is squeezed and fluid which is within the core returns to the topsheet surface causing a wet feeling for the user. Formed film topsheets were invented to avoid rewet. U.S. Pat. No. 3,929,135 to Thompson was the first to describe a phobic polymer impermeable web transformed into a network of interconnected capillaries with a specific range and design of taper to be used as a topsheet that would adequately transmit the fluid into the absorbent core.

Another topsheet found particularly functional for avoiding rewet is described in U.S. Pat. No. 4,324,246 to Mullane. Here, a phobic polymer is formed into a three dimensional apertured web having very specific parameters of Equivalent Hydraulic Diameter, loft, and open area.

Later it was learned, though, that most versions of phobic formed films would not perform well with respect to the necessary fluid acquisition rates needed for functional absorbent devices. If a topsheet cannot acquire a fluid insult at an appropriate rate, the diaper can leak in the leg area. U.S. Pat. Nos. 4,351,784, 4,456,570 and 4,535,020 to Thomas describe how a philic surface enhances fluid acquisition performance without sacrifice to the already known prevention of rewet.

U.S. Statutory Invention Registration H1,670 to Aziz describes how a formed film topsheet material can be placed between the core and the topsheet, more particularly a nonwoven topsheet, to prevent rewet when the nonwoven topsheet alone will not prevent rewet. WO 92/18078 to Colbert and U.S. Pat. No. 5,352,217 to Curro teach the use of two layers of perforated film in order to further prevent rewet. In a coassigned, copending application Ser. No. 09/668,649 a new innovative material useful as an acquisition distribution layer is described, said material having a high void volume space sufficient for the lateral distribution of fluids from repeated insults while keeping the skin of the user virtually free from any wetness sensation.

The current invention is an absorbent article with a more effective topsheet to acquisition distribution layer relationship that produces a drier feel to the user and generally better performance.

### SUMMARY OF THE INVENTION

An absorbent article may have topsheet, an absorbent core, and an acquisition distribution layer disposed between the topsheet and the core. The topsheet typically has a sufficient affinity to water and is therefore somewhat philic, but is not phobic. The acquisition distribution layer is more philic than the topsheet, thereby creating a surface energy gradient between the topsheet and the acquisition distribution layer.

The topsheet is preferably a formed film, though many nonwovens, particularly those typically known for use as topsheets, will also function adequately. The formed film acquisition distribution layer preferably has a high void volume. Other varieties of formed films with less void volume will also function adequately. The level of affinity to water of either the topsheet or the acquisition distribution layer may be created using surfactants, corona treatment, material selection or a combination of the above.

### BRIEF DESCRIPTION OF THE DRAWINGS

FIG. 1 is a cross sectional view of a typical absorbent article with a formed film layer between the topsheet and the core.

FIG. 2 is a cross sectional view of a formed film.

FIG. 3 is a plan view of the body facing side of a high void volume formed film used as an acquisition distribution layer.

FIG. 4 is a cross sectional view of a typical absorbent device with a high void volume formed film layer between the topsheet and the core.

FIG. 5 is a perspective view of the wire basket used to test the surface energy of various materials.

### DETAILED DESCRIPTION OF THE EXEMPLARY EMBODIMENTS OF THE INVENTION

This invention relates to absorbent articles having a three dimensional apertured formed film acquisition distribution layer. Examples of absorbent articles include diapers, incontinent articles, sanitary napkins, and similar articles.

Definitions:

For purposes of this application, the term “absorbent article” will refer to articles that absorb and contain fluids, such as body exudates. More specifically, the term refers to articles which are placed against or in proximity to the body of a wearer for absorbing and containing various exudates discharged from the body. The term “absorbent article”, as used herein, is intended to include diapers, incontinent articles, sanitary napkins, pantiliners, wound dressings, surgical drapes and other articles used to absorb body exudates.

The term “diaper” refers to a garment typically worn by infants and incontinent persons that is drawn up between the legs and fastened about the waist of the wearer. Examples of diapers from the prior art include diapers described in U.S. Pat. Re. No. 26,152, issued to Duncan, et al. on Jan. 31, 1967; U.S. Pat. No. 3,860,003 issued to Buell on Jan. 14, 1975; U.S. Pat. No. 4,610,678 issued to Weisman, et al. on Sep. 9, 1986; U.S. Pat. No. 4,673,402 issued to Weisman, et al. on Jun. 16, 1987; U.S. Pat. No. 4,695,278 issued to Lawson on Sep. 22, 1987; U.S. Pat. No. 4,704,115 issued to Buell on Nov. 3, 1987; U.S. Pat. No. 4,834,735 issued to Alemany, et al. on May 30, 1989; U.S. Pat. No. 4,888,231 issued to Angstadt on Dec. 19, 1989; and U.S. Pat. No. 4,909,803 issued to Aziz, et al. on Mar. 20, 1990.

The term “incontinent article” refers to diapers, inserts, pads, undergarments, e.g., pads held in place by a suspension system, such as a belt, or other device, inserts for absorbent articles, capacity boosters for absorbent articles, briefs, bed pads, and similar devices, whether worn by adults or other incontinent persons. Examples of incontinent articles include those disclosed in U.S. Pat. No. 4,253,461 issued to Strickland, et al. on Mar. 3, 1981; U.S. Pat. Nos. 4,597,760 and 4,597,761 issued to Buell; the above-mentioned U.S. Pat. Nos. 4,704,115; 4,909,802 issued to Ahr, et al.; U.S. Pat. No. 4,964,860 issued to Gipson, et al. on Oct. 23, 1990; and in U.S. patent application Ser. Nos. 07/637,090 and 07/637,571 filed respectively by Noel, et al. and Feist, et al. on Jan. 3, 1991.

The term “sanitary napkin” refers to an article that is worn by a female adjacent to the pudendal region that is intended to absorb and contain various exudates which are discharged from the body, e.g., blood, menses, and urine. Examples of sanitary napkins are disclosed in U.S. Pat. No. 4,285,343, issued to McNair on Aug. 25, 1981; U.S. Pat. Nos. 4,589,876 and 4,687,478, issued to Van Tilburg on May 20, 1986 and Aug. 18, 1987 respectively; U.S. Pat. Nos. 4,917,697 and 5,007,906 issued to Osborn, et al. on Apr. 17, 1990 and Apr. 16, 1991, respectively; and U.S. Pat. Nos. 4,950,264, and 5,009,653 issued to Osborn on Aug. 21, 1990 and Apr. 23, 1991, respectively; and in U.S. patent application Ser. No. 07/605,583 filed Oct. 29, 1990 in the name of Visscher, et al.

The term “pantiliner” refers to absorbent articles that are less bulky than sanitary napkins that are generally worn by women between their menstrual periods. Examples of pantiliners are disclosed in U.S. Pat. No. 4,738,676 entitled “Pantiliner” issued to Osborn on Apr. 19, 1988.

The term “gradient” shall mean any significant difference in affinity to water. This application focuses on a gradient between the topsheet and the acquisition distribution layer. As shown in Curro U.S. Pat. No. 5,352,217 and Colbert WO 92/18078 a gradient effect may be created where the topsheet aperatures, or capillary radius, was greater in size than those of the second layer beneath the topsheet. However, in the present invention a gradient is created by the gradient difference in surface energy between the two materials. In the preferred embodiment of the present invention, the apertures of the acquisition distribution layer are larger than the apertures of the topsheet, in direct conflict with the teachings of Curro and Colbert.

As used herein, the terms “phobic” and “philic” will mean hydrophobic and hydrophilic, respectively. These terms relate to a material's affinity to water. A phobic material has a low affinity to water and water tends to bead on the surface. Philic material has a high affinity for water and tends to spread on them. Whether a material is phobic or philic is typically determined by testing the surface contact angle of a drop of water on the surface of the material. If the film is phobic the water will form a tall bead and the contact angle is very great. If the film is philic the water will have affinity to the surface and flatten out, forming a very small angle. This test is ASTM D 5946-99 and is very well known in the industry.

Unfortunately, to run the water contact angle test the film must have a continuous uniform flat surface. Formed films are neither uniform nor flat. The three dimensional shape of the films does not provide sufficient flat area for the water contact angle method. If one attempts to flatten the sample between heated plattens, the surfactant, which has a lower viscosity than the polymer, may be displaced and therefore a true indication of the characteristics of the formed film itself is not represented.

Hence, another test has been devised—the Basket Sink Time Test based on European Disposables and Nonwovens Association (EDANA) ERT 10.3-99. In this test a small basket of approximately 50 mm in diameter and approximately 80 mm in length made of thin copper wire is utilized. The film to be tested is cut into rectangular shaped layers totaling 5 grams of weight, and then it is rolled and inserted into the basket. The affinity to water is measured by the rate at which the basket of material sinks in tap water. If the film is philic, its surface affinity for fluid will draw the fluid into the voids and air pockets existing in-between the many rolled up layers of the material, whether it is a nonwoven or a three dimensional apertured film. The higher the affinity to water, the faster a material will sink. The lower the affinity to water, the slower it will sink. An untreated polyolefin formed film, which is phobic, will not sink. For purposes of this application, a material is deemed to be “phobic” if it has a basket sink time greater than or equal to 120 seconds, while a material is deemed to be “philic” if it has a basket sink time less than 120 seconds. This test is described in more detail below.

The disclosures of all patents, patent applications and any patents which issue therefrom, as well as any corresponding published foreign patent applications, and publications mentioned throughout this patent application are hereby incorporated by reference herein. It is expressly not admitted, however, that any of the documents incorporated by reference herein teach or disclose the present invention. It is also expressly not admitted that any of the commercially available materials or products described herein teach or disclose the present invention.

Absorbent Article:

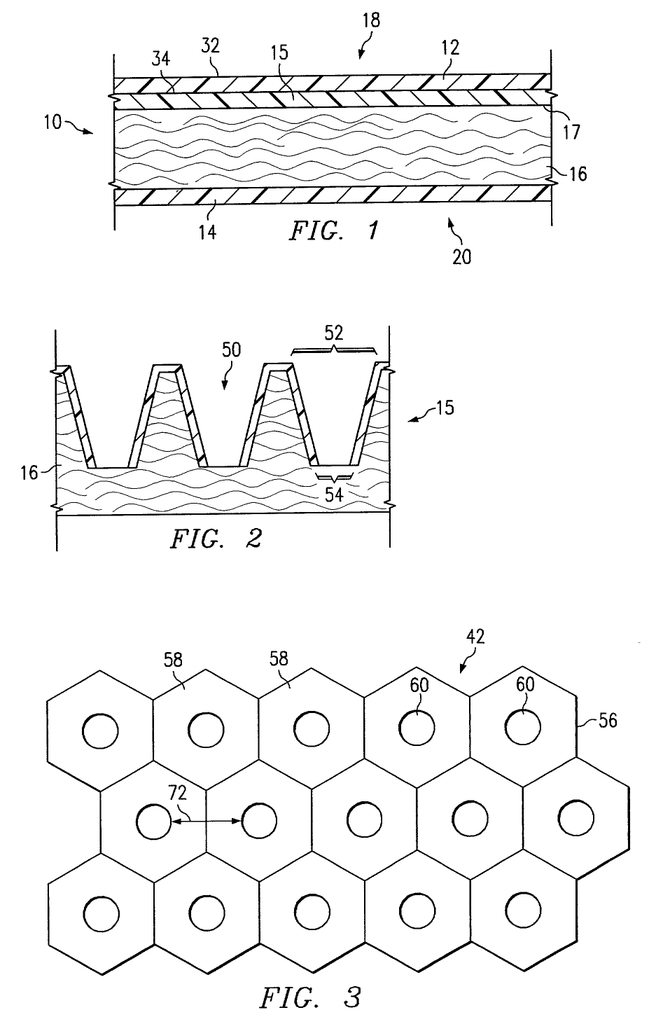

Referring now to FIG. 1, a simplified cross sectional representation of a typical absorbent article 10 is shown. It should be understood, however, that FIG. 1 is shown for purposes of example only, and should not be construed to limit the particular type or configuration of absorbent article. As shown in FIG. 1, absorbent article 10 basically comprises topsheet 12, backsheet 14, an acquisition distribution layer 15, and an absorbent core 16. Absorbent core 16 has a top or body facing side 17.

The absorbent article 10 has two surfaces, a body-contacting surface or body surface 18 and a garment-contacting surface or garment surface 20. The body surface 18 is intended to be worn adjacent to the body of the wearer. The garment surface 20 (FIG. 1) of the absorbent article 10 is on the opposite side and is intended to be placed adjacent to the wearer's undergarments or clothing when the absorbent article 10 is worn.

The individual components of the absorbent article 10 will now be looked at in greater detail. Topsheet 12 is compliant, soft-feeling and non-irritating to the wearer's skin. Further, topsheet 12 is apertured and thus liquid permeable, permitting liquids to readily penetrate through its thickness. The topsheet 12 has a body-facing side 32 (FIG. 1) and a garment-facing side 34 (FIG. 1). Absorbent core 16 has a top or body facing side 17. Throughout the remainder of this application, similar components will share the same numbers for all embodiments of the invention, e.g., “topsheet” will be designated by the numeral 12 in each embodiment.

Topsheet 12 is preferably made of an apertured formed film layer but may also be a nonwoven, a perforated nonwoven, or a perforated composite of nonwoven and film. Topsheet 12 is preferably bonded to acquisition distribution layer 15 (FIG. 1), although in some embodiments, topsheet 12 is not bonded to but instead lays in contact with acquisition distribution layer 15. Importantly, acquisition distribution layer 15 is between the topsheet 12 and absorbent core 16.

The topsheet 12 may be any geometrical shape and design, in plan view of the selected pattern or in cross-sectional view of the cell shape, previously known in the art as this aspect of the topsheet is not as critical as the level of surface energy of the formed film.

Formed Film Acquisition Distribution Layers:

Preferably, plastic film acquisition distribution layer 15 is an apertured thermoplastic film with tapered capillaries which has a run off percent of less than about 10 percent and which has an increased liquid flow rate through the tapered capillaries. The method of making such a film includes a two-fold surface treatment, which is taught by U.S. Pat. Nos. 4,535,020 and 4,456,570 to Thomas et al. entitled, “Perforated Film” and “Treatment of Perforated Film”, respectively. U.S. Pat. Nos. 4,535,020 and 4,456,570 are incorporated herein by reference. The method teaches that one surface treatment is provided by adding an internal chemical additive, namely a surfactant, to a film forming polyolefin resin. The additive is compounded or otherwise mixed or blended with the resin prior to the film being formed from the resin. After the film is formed, the other surface treatment is accomplished by treating the film with a corona discharge treatment which acts on the chemical additive to provide the apertured film with a zero or near zero percent run off. It should be understood that either the corona treatment alone or the resin added migrating surfactant alone can also be applied. Particularly with some of the newer surfactants, the synergistic effect of the combination of corona treatment and the resin added surfactant is less dramatic.

The apertured plastic film 15 is typically located between the topsheet 12 and the absorbent core 16. As shown in FIG. 2, the apertured plastic film 15 is a three-dimensional structure having a plurality of tapered capillaries 50, each of which has a base opening 52, and an apex opening 54. The apex of the openings 54 are in intimate contact with the absorbent core 16.

The apertured plastic film 15 is typically manufactured from a liquid impervious, thermoplastic material. One suitable material is a low density polyethylene film having an average calculated thickness of from about 0.0005 to 0.0045 inches (0.00127 to 0.0114 cm.). The thermoplastic material for use in the manufacture of a typical apertured plastic film 15 is selected from a group consisting generally of polyethylene, ethylene copolymers, polypropylene, propylene copolymers, polyvinyl chloride, starch base resins, polyvinylalcohol, polyurethanes, polyesters, cellulosics, ionomers, nylons, or combinations thereof.

In one typical embodiment, the thermoplastic material is provided with a multiplicity of tapered capillaries 50 in a manner, size, configuration, and orientation set forth in U.S. Pat. No. 3,939,135 issued to Thompson on Dec. 30, 1975. Other typical apertured plastic films are disclosed in U.S. Pat. No. 4,324,246, issued to Mullane, et al. on Apr. 13, 1982, U.S. Pat. No. 4,342,314, issued to Radel, et al. on Aug. 3, 1982, and U.S. Pat. No. 4,317,792 to Raley et al. on Mar. 2, 1982. The apertured plastic film 15 can also consist of other types of apertured plastic films. The type of film used depends on the type of processing that the film is subjected to during the manufacture of the topsheet 12. Other types of apertured films include, but are not limited to hydro-formed films. Hydro-formed films are described in at least some of the following U.S. Pat. Nos. 4,609,518, 4,629,643, 4,695,422, 4,772,444, 4,778,644, and 4,839,216 issued to Curro, et al., and U.S. Pat. No. 4,637,819 issued to Ouellette, et al.

Typically, the topsheet 12 and the apertured plastic film 15 are placed into a face-to-face relationship. The two components may be secured or unsecured. The two components, if secured, may be secured to each other by various methods. Typical methods for securing the nonwoven fabric and the apertured film 15 include, but are not limited to adhesives, fusion including heat bonding and/or pressure bonding, ultrasonics, and dynamic mechanical bonding such as crimping. Typically the acquisition distribution layer will not cover the same square area of the full diaper surface, but rather a more central, smaller area. Also, in preferred embodiments the acquisition layer is affixed or partially affixed to the contacting surface of the topsheet and the core, but is not affixed to the perimeter or edges, and particularly it is not directly affixed in any manner to the backsheet.

The adhesives can be applied in a uniform continuous layer, a patterned layer, or an array of separate lines, spirals, beads, or spots of adhesive. The adhesive attachment typically comprises an open pattern network of filaments of adhesive such as is disclosed in U.S. Pat. No. 4,573,986 issued to Minetola, et al. on Mar. 4, 1986, or an open pattern network of filaments having several lines of adhesive filaments swirled into a spiral pattern as illustrated by the apparatus and method shown in U.S. Pat. No. 3,911,173 issued to Sprague, Jr. on Oct. 7, 1975; U.S. Pat. No. 4,785,996 issued to Zieker, et al. on Nov. 22, 1978; and U.S. Pat. No. 4,842,666 issued to Werenicz on Jun. 27, 1989. Another method of heat/pressure bonding that could be used is described in U.S. Pat. No. 4,854,984 issued to Ball, et al. on Aug. 8, 1989.

The topsheet 12 and the apertured plastic film 15 can alternatively be integrally formed into a composite structure, as taught by Merz et al. in U.S. Pat. No. 4,995,930. The terms “composite”, “composite structure” or “combination”, as used herein, refer to relationships in which portions of the topsheet 12 are bonded to the film 15, and vice versa so that they are integrally attached.

High Void Volume Acquisition Distribution Layers:

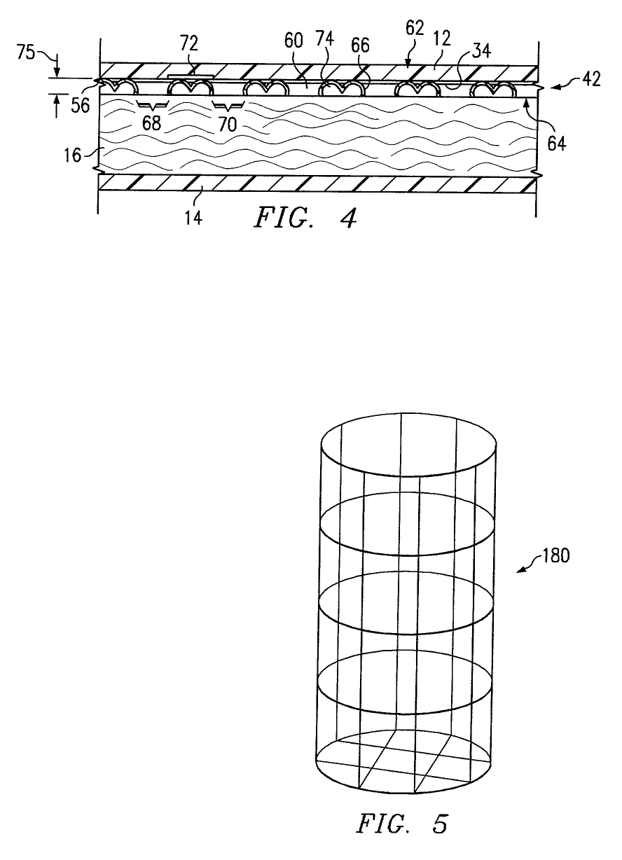

Referring now to FIGS. 3 and 4, a first embodiment of an improved absorbent article of the applicant's invention utilizes an acquisition distribution layer 42 made of a three dimensional apertured film 56 imparted with a hexagonal pattern. Although a hexagonal pattern is used for purposes of illustration, it should be understood that other patterns may also be used for any of the films described herein. Examples of other patterns include circular, oval, elliptical, polygonal, or other suitable patterns or combinations of patterns. The hexagonal pattern forms a plurality of adjacent hexagons or cells 58. In a preferred embodiment, the hexagonal pattern is based on a 8.75 mesh wherein “mesh” is the number of cells 58 aligned in a one-inch length. Although a mesh count of 8.75 is preferred, a mesh count of from 2 to 40 or more preferably from 4 to 25 may be used. Preferably, each cell 58 is provided with an aperture 60 that has a large hole diameter, e.g., 59 mils, which is large enough to allow insult fluids to be acquired through the three dimensional apertured film 56 as rapidly as the fluids are delivered.

Referring in particular to FIG. 4, which shows an enlarged cross sectional view of film 56 taken along line 5-5 of FIG. 3, three dimensional apertured film 56 has a body facing side or female side 62 and a garment facing side or male side 64. The garment-facing side 34 of the topsheet 12 is preferably maintained in close contact with the female side 62 of the apertured plastic film 56. Preferably topsheet 12 maintains contact with film 56.

As can be seen in FIG. 4, the film 56 is located between a topsheet 12 and an absorbent core 16. The apertured plastic film 56 is a three-dimensional structure having a plurality of capillaries 66, each of which has a base opening 68 and an apex opening 70. The apex openings 70 of the capillaries 66 are in intimate contact with the absorbent core 16, and preferably apex openings 70 are affixed to core 16 to insure this intimate contact. It should also be noted that essentially only the apex openings 70 of the capillaries 66 are in intimate contact with the core 16, thereby assuring that the void spaces 74 providing for lateral spillage remain substantially unencumbered. A land area 72 is formed between adjacent apertures 60 on the female side 62 of the apertured plastic film 56. A void volume space 74 (FIG. 4) is formed on the male side 64 of the apertured plastic film 56 that provides a fluid passageway between each of the cells 58. Preferably, the ratio of void volume space 74 versus apex opening space 70 is 2:1. The three dimensional apertured film 56 has a loft 75, i.e. the distance between the surface on the female side 62 and the planar surface on the male side 64, of from 0.0175″ to 0.125″, more preferably 0.035″ to 0.100″, and most preferably of 0.050″. The apertured plastic film 56 preferably has a density in the range of from about 0.890 g/cc to 0.990 g/cc, with the more preferred range of densities being from about 0.920 g/cc to 0.960 g/cc. The apertured plastic film 56 preferably has a melt indices in the range of from about 0.10 to about 15.0, with the more preferred range typically being from about 1.5 to about 8.5.

Surface Energy Gradients:

Topsheets 12 of formed films or nonwovens have long been treated with surfactants. The amount of surface energy of the topsheet is critical only in that the fluid will bead up on a purely phobic surface which might cause a diaper to leak, for example. Corona treatment alone has been proven effective in creating a philic formed film as taught in Thomas, U.S. Pat. No. 4,351,784 or preferably, migratable surfactants have been used as taught by Thomas, U.S. Pat. No. 4,456,570. Topsheets can also be coated with surfactants by spraying, rolling, or other known coating means.

However, these same known methods, when applied to acquisition distribution layers 15 yielded adequate performance for acquisition rate and rewet values but did not yield superior performance. Despite the teachings of the prior art, applicant found that by using a somewhat philic topsheet 12 in conjunction with a more philic acquisition distribution layer 15 the best performance is achieved.

In the preferred embodiments, the topsheet 12 is a formed film as described in Raley U.S. Pat. No. 4,317,792, Mullane U.S. Pat. No. 4,324,246 and/or Radel U.S. Pat. No. 4,342,314 and the acquisition distribution layer 15 is a high void volume type as described above. In alternative embodiments topsheet 12 may also be a nonwoven and acquisition distribution layer 15 may be a formed film of Thompson U.S. Pat. No. 4,292,135, Raley U.S. Pat. No. 4,317,792, Mullane U.S. Pat. No. 4,324,246 and/or Radel U.S. Pat. No. 4,342,314, or any similar material. All acquisition distribution layer 15 types in combination with all topsheet 12 types can benefit from the surface energy gradient of this invention. In the formed film topsheet 12 of the preferred embodiment a surfactant sold by Atmer Corporation and coded Atmer 100 was incorporated at 1,500-10,000 ppm in a formed film similar to that described in the Raley '792 patent. More preferably 4,000-8,000 ppm is used, and most preferably 7,000 ppm. This topsheet 12 incorporating 6080 ppm of Atmer 100 has an average Basket Sink Time of 45.2 seconds. In other functional embodiments a philic treated 17 gsm spun bonded polypropylene nonwoven topsheet sold by BBA Corporation of Simpsonville, S.C. is used. This topsheet has a basket sink time of 9.5 seconds.

The acquisition distribution layer 15 of the preferred embodiment has a resin blended migrating surfactant sold by Techmer, coded PM11930E63, added at 10,000-30,000 ppm. In the preferred embodiment an acquisition distribution layer containing 20,00 ppm surfactant has a Basket Sink Time of 3.5 seconds. This high void volume acquisition distribution layer 15 is known as AquiDry^{™} ADL and is sold by Tredegar Film Products Corporation. For reference, a film similar to that described in Raley '792 without the addition of a surfactant or similar treatment was deemed phobic because it did not sink in less than two minutes (120 seconds).

Test Data

Basket Sink Time Test:

As discussed above, the typical test for water affinity or surface energy can not be effectively carried out on formed film samples. Therefore, the Basket Sink Time test was used as an alternative. The test method is described in general in EDANA 10.3-99, but has been modified slightly for this application. The method involves rolling the piece of material to be tested (in the manner set forth below) and placing the material into a wire basket 180, as shown in FIG. 5. The wire basket 180 is then dropped from a height of approximately 25 mm into a sufficiently large container of tap water. The time is recorded from the moment the basket 180 is released until the basket 180 is fully submerged. If that time is equal to or greater than two minutes then the material is phobic and the timing is stopped.

The material being tested must be aged for at least 24 hours in a standard laboratory environment of 23 degrees Celsius, plus or minus 2 degrees, and 50% relative humidity, plus or minus 5%.

The basket 180 is a cylindrical wire basket with an open end. The diameter of the basket 180 is approximately 50 mm and the length is approximately 80 mm. The basket 180 is made of approximately 0.5 copper wire and constructed with a target mass of 3 g. Novi Profibre of France is a vendor of such baskets 180.

The material to be tested is cut into a piece measuring 76 mm in width and long enough to achieve a mass of 5 grams. The material is rolled neatly and loosely with the female side of the film out and placed into the basket 180. The tap water should acclimate to the surrounding laboratory temperature prior to testing, and therefore be at a temperature of 23 degrees Celsius, plus or minus 2 degrees. The basket 180 containing the material is positioned 25 mm from the surface of the tap water. The basket 180 is dropped into the water, the timing commencing upon release. Once the basket 180 is completely submerged under the surface of the water the time is recorded. If two minutes elapse end that test and record >120 seconds. Repeat with two more samples of the same material. The three times are averaged to determine the BST, Basket Sink Time.

For the purposes of averaging the results of tests where the sample is close to the 120 second mark, it may be necessary to time certain samples beyond 120 seconds. For example, if a test is less than 120 seconds and a later test reaches 120 seconds, then the later test will be recorded past 120 seconds so that the later test may be averaged with the earlier test. Likewise, if a test is greater than 120 seconds and a later test is less than 120 seconds, then the test of greater than 120 seconds will be discarded and another test will be run to average with the test that was less than 120 seconds. Importantly, where the results are near 120 seconds, the necessary test data will be collected to calculate the philicity of the material. This sort of averaging was not encountered in applicants test collection relating to this application.

The assignee of this invention has filed for a registered trademark “AquiDry” for their acquisition distribution layer (ADL). The term, when used, will designate the innovative high void volume formed film material.

The following data was collected using this method:

| Material | BST (seconds) |
|---|---|
| Raley, no treatment | >120 |
| Raley, Atmer 100 | 45.2 |
| Philic Nonwoven | 9.5 |
| AquilDry ™ ADL | 3.5 |

As can be seen from the above, both the Raley topsheet 12 treated with Atmer 100 and the philic treated nonwoven topsheet 12 are somewhat philic, but the AquiDry™ ADL 15 is more philic. This differential in surface energy improves acquisition rate and rewet properties. Specifically, and most importantly, rewet properties were improved on a repeated insult (2nd & 3rd) occurrence. The liquid is drawn to the acquisition distribution layer and spread more rapidly and evenly by the acquisition distribution layer due to the surface energy gradient between the topsheet 12 and acquisition distribution layer 15.

While the most preferred embodiment of this invention is the use of a high void volume acquisition distribution layer 15, beneficial effects may be achieved with earlier versions of formed films being placed in the acquisition distribution layer 15 position as well. Likewise, while a most preferred embodiment uses a formed film topsheet 12, nonwoven and other topsheets 12 may benefit from this invention as well.

From the above, it will be appreciated that applicant's invention will reduce or eliminate the wetness sensation felt by the user during and after repeated insults as unabsorbed fluid flows from an area of saturated core material to an area of unsaturated core material for absorption. Applicant's invention redirects unabsorbed fluids to non-saturated areas of a core material 16 while preventing substantial contact of the unabsorbed fluids with the topsheet 12. The invention of the applicant prevents an unpleasant feeling of wetness of the topsheet 12 while providing the ability to receive multiple insults at a singular point.

Strikethrough and Rewet Test:

Strikethrough is a measure of the fluid acquisition rate of an absorbent article. Strikethrough is represented in a time, typically in seconds. A lower value for strikethrough is preferable. Rewet is a measure of the amount of fluid which comes to the surface of an absorbent article when pressure is placed on the absorbent article. Rewet is measured in weight, typically in grams, of fluid absorbed at the surface when pressure is placed on the absorbent article. The following test procedure was used to collect data for this application.

The test consists of providing multiple insults (typically 3-5) at an insult point, each insult being of a set volume of liquid. The time required to absorb the liquid is the strikethrough. After the liquid has been absorbed, then the absorbent article is pressed under a set amount of weight with dry absorbent papers. The absorbent papers are weighed to calculate the rewet value. This procedure is described in more detail below.

An absorbent article is unfolded with the topsheet side facing up and taped down with masking tape to a lab table such that any elastic legs are upright to prevent fluid flow off of the flattened absorbent article. The insult point is chosen on the article where the tests will be conducted. For a baby diaper, the insult point is six inches back from the front end of the absorbent core and centered on the narrow dimension of the core. For an adult incontinent brief the insult point is 13 inches back from the front end of the core and centered on the narrow dimension of the core.

A preparation of 70 dyne saline solution is prepared to test the strikethrough and rewet of the absorbent articles. The amount of the solution used in each test depends on the type of absorbent article being tested. For baby diapers, 80 ml per insult is used. For adult incontinent briefs, 160 ml is used.

A test cylinder having an inner-diameter of 51 mm and a height of 100 mm is centered on the insult point. The predetermined amount of saline solution is poured into the test cylinder. A stopwatch is started when the liquid contacts the sample's topsheet surface. The stopwatch is stopped when no liquid remains in the bottom of the 51 mm cylinder. The time on the stopwatch at this point indicates the strikethrough time for the first insult.

The cylinder is then removed from the sample and an elapsed time of 10 minutes is allowed to pass to allow the saline solution to be absorbed into the core of the sample. After 10 minutes has passed, a stack of 10, 4″×4″ dry absorbency filter papers, which has previously been weighed in its dry state, is placed over the insult point. An 8 lb., 4″×4″ weight is then gently placed on top of the stack. The weight is allowed to remain on top of the stack of filter paper for two minutes at which point the weight is removed and the now wet filter paper is removed and weighed. The dry weight of the filter paper is subtracted from the wet weight of the filter paper to determine the rewet value in grams for the first insult.

A second insult follows the same procedure as the first insult and is performed on the same absorbent articles. In other words, the 51 mm inner-diameter cylinder is again centered on same insult point, the predetermined amount of saline solution is poured into the cylinder onto the sample, and a stopwatch is started to measure the strikethrough time.

After the fluid has been absorbed, and the strikethrough time for the second insult is recorded, the cylinder is removed from the sample, the stopwatch is reset and 10 minutes are allowed to elapse. After the 10 minutes have elapsed, a stack of 20 sheets of 4″×4″ dry absorbency filter paper is placed over the insult point. Again, an 8 lb, 4″×4″ weight is gently placed on top of the stack. This stack of absorbency paper and an 8 lb weight is allowed to stay on top of the sample for two minutes. After two minutes, the weight and the now wet filter paper is removed, the filter paper is weighed in its wet state, and the previously measured dry weight of the filter paper is subtracted from the wet weight to calculate a rewet value for the second insult.

This test, as described above, is carried out on several samples of each type of absorbent article. The tests are then averaged over the several samples to determine an average strikethrough and average rewet value for each insult tested. Furthermore, this test can be adjusted to be effective on a variety of absorbent articles, ranging from baby diapers to adult incontinent articles by adjusting the volume of saline solution used in each insult from between 80 ml to approximately 160 ml. The 4″×4″ 8 lb weight is designed to produce approximately 0.5 psi on the surface of the absorbent article. The bottom of the weight has a poly-covered foam pad to reduce sharp differences in how the weight is applied. The 51 mm inner-diameter is unweighted and therefore typically manufactured of plastic, but could also be manufactured of other lightweight materials such that the weight of the cylinder does not affect the absorbency of the absorbent article. The 70 dyne saline solution may have a variance of surface tension in the range of plus/minus 5 dynes. Typically a 0.9% NaCl (saline) solution is used to achieve 70 dynes (+/−5 dynes) as required. The saline solution should be prepared ahead of time to ensure that it is at laboratory ambient temperature, approximately 75° F., at the time of the test.

The following table of data demonstrates how on a second repeated insult occurrence, acquisition rate and rewet values for articles using the claimed invention are superior over prior art.

| | | | Second Insult Results | |
|---|---|---|---|---|
| Topsheet | | Gradient | Acquisition | |
| Type | ADL Type | Yes, No | Rate, Sec | Rewet gm |
| Nonwoven | None | No | 46.05 | 14.23 |
| Nonwoven | Raley FF | No | 28.54 | 6.56 |
| Nonwoven | AquilDry ™ FF | Yes | 22.31 | 2.52 |
| Raley FF | Raley FF | Yes | 24.60 | 0.24 |
| Raley FF | AquilDry ™ FF | Yes | 20.28 | 0.09 |

The present invention is illustrated herein by example, and various modifications may be made by a person of ordinary skill in the art. For example, various geometries, materials and multiple-layer film combinations fall within the scope of the invention. As another example, although the present invention has been described in connection with diapers, incontinent articles, sanitary napkins, and related products, the absorbent articles of the present invention are fully applicable to other, similar products, including, without limitation, other body coverings where absorbent materials may be desired. Such body coverings may include medical drapes, medical gowns, medical smocks, ostomy appliances, feminine hygiene products, body transfer sheets, fluid collection pouches, industrial clean room garments and other products.

It is therefore believed that the present invention will be apparent from the foregoing description. While the methods and articles shown or described have been characterized as being preferred it should be obvious that various changes and modifications may be made therefrom without departing from the spirit and scope of the invention as defined in the following claims.

We claim:

1. An absorbent article comprising:
a topsheet which is philic;
an absorbent core; and
an acquisition distribution layer between the topsheet and the absorbent core which is more philic than the topsheet.

2. The absorbent article of claim 1 wherein the topsheet is an apertured formed film.

3. The absorbent article of claim 1 wherein the topsheet is a nonwoven.

4. The absorbent article of claim 1 wherein the acquisition distribution layer is an apertured formed film.

5. The absorbent article of claim 4 wherein the acquisition distribution layer has a high void volume.

6. The absorbent article of claim 1 wherein:
the topsheet is a nonwoven; and
the acquisition distribution layer is a high void volume apertured vacuum formed film.

7. The absorbent article of claim 1, wherein the topsheet has a basket sink time of about 9 seconds to about 120 seconds.

8. The absorbent article of claim 1, wherein the acquisition distribution layer has a basket sink time of less than about 20 seconds.

9. The absorbent article of claim 1, wherein:
the topsheet has a basket sink time of about 9 seconds to about 120 seconds; and
the acquisition distribution layer has a basket sink time of less than about 9 seconds.

10. The absorbent article of claim 1, wherein the topsheet has a basket sink time of about 9 seconds to about 60 seconds.

11. The absorbent article of claim 1, wherein the acquisition distribution layer has a basket sink time of less than about 10 seconds

12. The absorbent article of claim 1, wherein:
the topsheet has a basket sink time of about 9 seconds to about 60 seconds; and
the acquisition distribution layer has a basket sink time of less than about 9 seconds.

13. The absorbent article of claim 1 wherein:
the topsheet has a basket sink time less than 120 seconds; and
the acquisition distribution layer has a basket sink time at least 3 seconds less than the topsheet basket sink time.

14. The absorbent article of claim 1 wherein:
the topsheet has a basket sink time less than 120 seconds; and
the acquisition distribution layer has a basket sink time at least 30 seconds less than the topsheet basket sink time.

15. An absorbent article comprising:
an apertured formed film topsheet which is philic;
an absorbent core; and
an apertured formed film acquisition distribution layer between the topsheet and the core which is more philic than the topsheet.

16. The absorbent article of claim 15 wherein the acquisition distribution layer has a high void volume.

17. The absorbent article of claim 15, wherein the topsheet has a basket sink time of about 20 seconds to about 120 seconds.

18. The absorbent article of claim 15, wherein the acquisition distribution layer has a basket sink time of less than about 20 seconds.

19. The absorbent article of claim 15, wherein:
the topsheet has a basket sink time of about 20 seconds to about 120 seconds; and
the acquisition distribution layer has a basket sink time of less than about 20 seconds.

20. The absorbent article of claim 15, wherein the topsheet has a basket sink time of about 30 seconds to about 60 seconds.

21. The absorbent article of claim 15, wherein the acquisition distribution layer has a basket sink time less than about 10 seconds

22. The absorbent article of claim 15, wherein:
the topsheet has a basket sink time of about 30 seconds to about 60 seconds; and
the acquisition distribution layer has a basket sink time of less than about 10 seconds.

23. An absorbent article comprising:
a nonwoven topsheet which is philic;
an absorbent core; and
an acquisition distribution layer between the topsheet and the core which is more philic than the topsheet.

24. The absorbent article of claim 23 wherein the acquisition distribution layer is an apertured formed film.

25. The absorbent article of claim 24 wherein the acquisition distribution layer has a high void volume.

26. The absorbent article of claim 23, wherein the topsheet has a basket sink time of about 9 seconds to about 120 seconds.

27. The absorbent article of claim 23, wherein the acquisition distribution layer has a basket sink time of less than about 6 seconds.

28. The absorbent article of claim 23, wherein:
the topsheet has a basket sink time of about 9 seconds to about 120 seconds; and
the acquisition distribution layer has a basket sink time of less than about 6 seconds.

29. The absorbent article of claim 23, wherein the topsheet has a basket sink time of about 9 seconds to about 60 seconds.

30. The absorbent article of claim 23, wherein the acquisition distribution layer has a basket sink time less than about 4 seconds

31. The absorbent article of claim 23, wherein:
the topsheet has a basket sink time of about 9 seconds to about 60 seconds; and
the acquisition distribution layer has a basket sink time of less than about 4 seconds.
